(19) United States
(12) Patent Application Publication
(10) Pub. No.: US 2008/0214500 A1
(43) Pub. Date: Sep. 4, 2008

(54) Injectable cement composition for orthopaedic and dental use

(76) Inventors: Leif Hermansson, Uppsala (SE); Hakan Engqvist, Knivsta (SE)

Correspondence Address: YOUNG & THOMPSON, 209 Madison Street, Suite 500, ALEXANDRIA, VA, 22314, US

(21) Appl. No.: 11/712,413

(22) Filed: Mar. 1, 2007

(51) Int. Cl. A61K 31/695; B01F 1/00; C04B 35/44

(52) U.S. Cl. 514/63; 252/364; 501/135; 501/153

(57) ABSTRACT

The present invention relates to ceramic precursor powder compositions and chemically bonded ceramic (CBC) materials, Ca-aluminate and/or calcium silicate, and a composite biomaterial with prolonged shelf time of the precursor, suitable for orthopaedic applications with improved injectability. The present invention also relates to a method of manufacturing said cured material, bioelements, implants, or drug delivery carrier materials made by said cured material, a kit comprising the ceramic precursor powder and hydration liquid, as well as the use of said ceramic precursor powder and hydration liquid, or said cured material, for orthopaedic and dental applications.

### FIELD OF THE INVENTION

The present invention relates to ceramic precursor powder compositions and chemically bonded ceramic (CBC) materials, calcium aluminate- and/or calcium silicate-based ones, and composite biomaterials suitable for orthopaedic applications with improved injectability.

### BACKGROUND

Chemically bonded ceramics are formed from mixing ceramic precursor powder compositions with a water containing liquid. Generally the CBC precursor powders originate from the calcium silicate, calcium aluminate, calcium phosphate or calcium sulphate systems. The CBC precursor powder can be mixed with inert particles, so-called fillers, for various reasons, e.g. increased strength and dimensional stability. CBC systems intended for use in orthopaedic and dental applications are described e.g. in the Ph. D. thesis by M. Nilsson “Injectable calcium sulphate and calcium phosphate bone substitutes”, Lund University 2003, and the Ph. D. thesis by L. Kraft “Calcium aluminate-based cement as dental restoratives materials”, Uppsala University, 2002. General aspects of using CBC materials based on Ca-aluminates related to manufacturing, dimensional stability and mechanical strength in dental and orthopaedic applications have earlier been described, e.g. in U.S. Pat. No. 6,969,424 B2, WO 2004 37215, WO 2004 58124 and WO 2003 55 450.

The CBC precursor powder materials react with water to form the final CBC material. The hydrated material is described as being hydraulic, meaning that it is not further reactive to water. Being reactive to water or water vapour in the precursor powder form, also means that the humidity in the air potentially can be harmful to the powder, leading to that a pre-reacted or partly pre-reacted powder, which subsequently in the process may not be formed and used in the intended way. Such powder exhibits short shelf life and is difficult to mix and handle, and may not have the proper setting properties. The final strength of the hardened CBC material may also be negatively influenced by a prematurely reacted powder.

This problem is well-known in the cement industry, and where it is known that a relative humidity (RH) of above 70% results in a sub-optimal product. The reproducibility and packaging demands, however, are much higher for CBC precursor powders within dentistry and orthopaedic applications, where considerably finer precursor particles are required, and applying the same RH-limits as for traditional cements, causes problems.

Injectable ceramics for orthopaedic applications are formed from mixing ceramic precursor powder compositions with a water-containing liquid. Generally the precursor powders originate from the calcium phosphate cement system. Calcium phosphate cements (CPC) are used as injectable orthopaedic cements. The injectability of an orthopaedic material is very important since it gives the surgeon the possibility to choose needle size depending on the voids to be filled and also to have enough time for injection, i.e. how to control the time available for injection, the so-called working. This is especially important when working with minimally invasive techniques, where a thin needle results in a less invasive operation. Presently the CPC suffers from phase separation (between ceramic powder and hydration liquid) due to the shear force situation within the cement. This results in a paste which cannot be extruded through needles thinner than 11 gauge without extreme caution.

For vertebroplasty the radio-opacity during injection is, as mentioned above, very important. Normally for orthopaedic applications radio-opacity achieved by adding a an additive imparting radio-opacity to the precursor powder. One such example is barium sulphate powder. Adding such powders to CPC results in problems with viscosity of the mix and in greater difficulties to inject the material through thin needles.

Thus, there is a need for a ceramic bone replacement material that can be easily handled and injected using fine needles, without the material phase-separating, and which, when hardened, exhibits the proper strength characteristics, while being radio-opaque. There is also a need for controlled manufacturing, packaging and storage methods for such a material.

### BRIEF DESCRIPTION OF THE INVENTION

The present invention relates to a ceramic bone replacement material that possesses all of the above-mentioned properties, and which may suitably be used in orthopaedic applications, such as vertebroplasty. The present invention also relates to the manufacturing, packaging and storage conditions for hydraulic precursor powders upon which said ceramic bone replacement material is based.

The present invention describes a ceramic system that comprises a ceramic precursor powder and a hydration liquid, that when mixed, The proposed ceramic precursor powder may also comprise additives that impart of radio-opacity.

The above-mentioned advantageous properties are achieved by a ceramic system comprising a hydraulic ceramic precursor powder which is mixed with a specific hydration liquid, resulting in a paste that exhibits an increased handling and injectability (without phase-separation) compared to that of the CPC systems. When cured, said paste forms a ceramic material exhibiting a high strength. The ceramic precursor powder may optionally comprise additives (a high-density additive) imparting a high radio-opacity that improves the X-ray visibility for a user during injection.

The injectability of such systems allows the material to be injected even through 13 gauge needles or larger using both 1 ml syringes or using more developed delivery systems, such as for example the injection system described in the co-pending provisional U.S. application No. 60/784,085.

However, aspects of the precursor powder quality must be taken into account. Surprisingly the injectability can be controlled, not just by the added water through the hydration liquid, but by the water content in the precursor powder. If during manufacturing, said precursor powder contains too much water, as well as experience too high humidity during packaging, the subsequent handling properties are negatively affected, resulting in a decreased working time and setting time. In addition, the injectability is negatively influenced by such water content.

The amount of water in the precursor powder is according to the present invention controlled as regards the relative humidity during manufacturing and packaging of the powder. The present inventors have surprisingly found that if the amount of water exceeds a certain limit in the precursor powder, the described properties are negatively affected. The allowable water content may be measured by controlling the water content in the packaged precursor powder. The measured relative humidity in the precursor powder or water content (measured as loss on ignition) may then be used to determine the status of the precursor powder, and if the precursor powder is still “fit” for obtaining optimal properties. This discovery enables a user to determine if properties such as correct working time, setting time, and final strength of the ceramic material is still achievable.

The present invention also relates to a method of manufacturing said cured material, bioelements, implants, or drug delivery carrier materials based on said precursor powder or said cured material, a kit comprising the ceramic precursor powder and hydration liquid, as well as the use of said ceramic precursor powder and hydration liquid, or said cured material, for orthopaedic and dental applications.

The mechanisms of the chemical system used in this application is described more in detail in a separate patent application Ser. No. ______ , filed Mar. 1, 2007, which is incorporated herein by reference.

### DETAILED DESCRIPTION OF THE INVENTION

The present ceramic material allows a) the material to be delivered through thin needles, b) possesses high radio-opacity, and c) makes it possible to inject the material via an injection device or system.

In some situations, the orthopaedic surgeon needs to follow the injection of the material into the body under live-fluoroscopy. This is especially important for vertebroplasty, injection of material into a fractured vertebrae via a minimally invasive procedure, where possible leakage of material into the spinal column can be very dangerous for the patient. Injection is often performed with the surgeon's hand also under the fluoroscope, resulting in a high X-ray dose for the surgeon. In such a situation, the ceramic paste may be injecting using an injection system such as for example described in the co-pending provisional U.S. application No. 60/784,085, which allows the surgeon to stand outside the fluoroscope while injecting the material into a defect. However, such injection systems, combined with the overall difficulty of injecting materials through thin needles, put high demands on the biomaterial, and thus pose a problem.

The ceramic biomaterial comprises a powder and a hydration liquid, which are mixed just before usage. The mixing can be done manually, but is preferably performed using a mixing device. After mixing, the formed paste can be transferred to an injection device via a transfer device.

The precursor powder according to the invention comprises in a basic embodiment:
Calcium aluminate as hydraulic precursor
Micro-silica as precursor additive

Said precursor powder are mixed with the hydration liquid according to the invention, which comprises:

mixed with, LiCl and
water
methyl cellulose, and

polycarboxylic acid

More specifically, the components of the precursor powder have the following characteristics:

### Calcium Aluminate

The calcium aluminate may have a grain size of below 30 micrometer, preferably below 20 micrometer, and more preferably below 15 micrometer. The grain size is determined as d99 (99%<stated value) using laser diffraction and calculated from the volume distribution, i.e. 1% of the powder may be of greater grain size.

The calcium aluminate is to more than 70 atomic % comprised of CaO(Al₂O₃) and to less than 30 atomic % comprised of one or more of the phases (CaO)₁₂(Al₂O₃)₇, (CaO)₃Al₂O₃, CaO(Al₂O₃)₂, CaO(Al₂O₃)₆, and CaO(Al₂O₃) glass. The calcium aluminate constitutes 55-65 wt-%, preferably 57-63 wt-%, of the total amount of precursor powder. The calcium aluminate is the reactive phase (binder phase).

### Micro-Silica

The micro-silica (SiO₂) may have a grain size of below 30, preferably below 20 nm. The micro-silica is added in an amount of 0.5-5 wt-%, preferably 0.7-1.3 wt-%, of the total amount of the precursor powder.

The nano-size silica (SiO₂) could also be included in the hydration liquid.

Zirconium dioxide

Zirconium dioxide may optionally be added as an inert precursor additive for increased radio-opacity. The zirconium dioxide (ZrO2) may have a grain size of below 10 micrometer, preferably below 5 micrometer, determined as d99 (99%<stated value) using laser diffraction. The zirconium dioxide is added to achieve extra radio-opacity and is considered as a non-reacting, inert phase. The ZrO₂ is added in an amount of 35-45 wt-%, preferably 38-42 wt-%, of the total amount of the precursor powder. If radio-opacity is not required for a certain application, the ZrO₂ may also be mixed with or replaced by another inert filler material, in the same amounts and grain sizes.

### Optional Additives

Calcium Silicate

Calcium silicate may also be added to the precursor powder as an additional hydrating phase (also a reactive phase), in the form of C₃S or C₂S or combinations thereof, in the amount of below 10 wt-%. of the total amount of the precursor powder. The grain size should be below 40 micrometer, preferably below 20 micrometer. The calcium silicate may also replace the calcium aluminate phase.

More specifically, the components of the hydration liquid have the following characteristics:

### Water

90-95 wt-% preferably 92-94 wt-% of the hydration liquid is constituted by water.

### Polycarboxylic Compound

The polycarboxylic compound may have a molecular weight within the interval 10000-50000, and constitutes 3-5 wt-%, preferably 3.7-4.3 wt-% of the hydration liquid. The compound is added to control the viscosity of the paste.

### Methyl Cellulose

The methyl cellulose constitutes 1-5 wt-% of the hydration liquid, preferably 2.5-3.5 wt-%. The compound is added to control viscosity and cohesion of a paste.

### Lithium Chloride

Lithium chloride (LiCl) constitutes less than 0.2 wt-%, normally 0.05-0.2 wt-%, of the hydration liquid. LiCl is added to control the setting time.

When mixed, the precursor powder and the hydration liquid may form a paste or a thick slurry depending on the water-to-cement (liquid-to-powder) ratio. The powder-to-liquid (p/l) ratio should be kept within 3.75-5, preferably 4-4.5.

The components added to the liquid promote a high cohesiveness of the paste. This means that the paste is easily kept together during injection, thus avoiding e.g. phase separation. This reduces also the risk of uncontrolled spread of the paste into undesired voids, e.g. the spinal column.

The precursor powder may be kept at a relative humidity of below 60%, preferably below 50%, during manufacturing and packaging. If not the reactive calcium aluminate and/or calcium silicates start to react with the water in the air and the function of the powder is negatively affected. However, according to the present invention, it is also possible to measure if a ceramic precursor powder has experienced too high humidity during manufacturing and/or packing. This can be measured as the ignition loss, i.e. the amount of water evaporated from the powder if heated above a certain temperature, where the chemically bonded water is decomposed, typically at temperatures above 300 C. The critical ignition loss has been measured to 0.05% of the precursor powder weight. This ignition loss is related to the relative humidity of <60%.

During powder preparation, storage and handling of the precursor powder, temperatures of less than 25° C. may preferably be used, since this under normal conditions will not involve detrimental levels of relative humidity.

In order to protect the precursor powder, the present invention provides a precursor powder that is packaged and stored under vacuum and/or inert gas, e.g. nitrogen and/or argon. Said powder will feature a loss on ignition less than 0.08%. Such a powder may also be provided in a kit comprising the hydration liquid (stored separately)

### EXAMPLE 1

Tests were conducted to test the shelf life of precursor powder compositions as function of the relative humidity during packaging. The shelf life was evaluated according to working time and setting time measurements as described below.

### Material

The precursor powder, see Table 1, was packaged in capsules in clean room facilities with controlled RH. The hydration liquid was also filled in syringes in clean room facilities, under controlled RH. Before packaging, the precursor powder was homogenised using tumbling, and the hydration liquid was homogenised through mixing.

TABLE 1
Composition of the precursor powder and hydration liquid

| | | | GRAIN |
|---|---|---|---|
| | Chemical | Amount | SIZE |
| Compound | formula | [wt %] | [μm] |
| Precursor powder | | | |
| Calcium Aluminate | CaO•Al₂O₃ | 59 | <12 |
| Zirconium dioxide | ZrO₂ | 40 | <5 |
| μ-Silica | SiO₂ | 1 | 0.014 |
| Hydration liquid | | | |
| Water | H₂O | 93 | — |
| Polycarboxylic | MPEGMa | 4 | — |
| compound | | | |
| Methyl cellulose | MetC | 2.8 | — |
| Lithium chloride | LiCl | 0.2 | — |

### Experimental Set-Up

The precursor powder and hydration liquid were packaged under 30%, 40%, 50%, 60% and 70% RH and stored under room temperature and normal RH for 3, 6 and 12 months. 12 capsules and syringes for each RH-package condition and time period were tested regarding working time and setting time. Mixing of the precursor powder and liquid was performed using a machine mixer and a powder to liquid ratio of 4.2. The working time was evaluated as ejection time through 11 Gauge syringes at RT and setting time as the time at peak temperature during setting. The aim was to have a constant working time and setting time throughout the test period. This is important to the reproducibility in the handling of the material.

### Results

The results from the testing are presented in Table 2. The results show that for a precursor powder and liquid packaged at a RH 60% or below, the setting and working times were constant. For a precursor powder and liquid packaged at a higher RH, the working time and setting time was considerably extended.

TABLE 2
Working time and setting time as a function of storage time and
RH during packaging.

| | RH (%) during | Working | Setting |
|---|---|---|---|
| Storage Time | packaging | time | time |
| 0 | 30 | 5 | 12 |
| | 40 | 5 | 11 |
| | 50 | 5.2 | 12 |
| | 60 | 5 | 13 |
| | 70 | 8 | 17 |
| 3 months | 30 | 5.3 | 13 |
| | 40 | 4.9 | 12 |
| | 50 | 5.6 | 12 |
| | 60 | 7 | 14 |
| | 70 | 8 | 16 |
| 6 months | 30 | 5.1 | 12 |
| | 40 | 5.5 | 12 |
| | 50 | 5.3 | 13 |
| | 60 | 7 | 15 |
| | 70 | 7 | 17 |
| 12 months | 30 | 5 | 12 |
| | 40 | 5.3 | 11 |
| | 50 | 5.2 | 12 |
| | 60 | 7 | 15 |
| | 70 | 8 | 18 |

Another finding was that for a RH above 60%, the loss on ignition, which corresponds to the amount of chemically bonded water formed already in the storage period, was measurable, and above 0.02 weight-%, and up to 0.08 weight-%.

### Conclusions

Packaging at 60% RH or below assures a shelf-life of more than 12 months. Packaging at 70% RH prolongs the working time and setting time directly, i.e. already at packaging.

### EXAMPLE 2

A series of experiments was conducted to test the radio-opacity and injectability of the ceramic paste through needles. The pastes based on calcium aluminate cement were compared to pastes based on calcium phosphate cement.

### Materials

The calcium aluminate-based precursor powder had the composition as described in Table 1 above. The calcium phosphate-based precursor powder had the precursor powder composition (in wt. %): α-TCP (71%), Mg₃(PO₄)₂ (10%), MgHPO₄ (3.8%), SrCO₃ (3.6%) and ZrO₂ (10%) and the hydration liquid H₂O, (NH₄)₂HPO₄ (3.5M).

### Experimental Set-Up

A calcium aluminate precursor powder and hydration liquid were mixed using machine vibrator in a powder-to-liquid ratio of 4.2. The calcium phosphate powder and hydration liquid were mixed using machine vibrator in a powder-to-liquid ratio of 3.

Two comparable tests were conducted:
1. Injectability through 1 ml syringes and 11 or 13 Gauge needles directly after mixing.
2. Radio-opacity after hardening, 1 mm thick discs of hardened materials were manufactured and compared to 2 mm thick discs of Al in giving radio-opacity.

### Results

The calcium aluminate-based paste was possible to inject through both 11 and 13 Gauge needles. The calcium phosphate paste was not possible to inject through neither of the needle sizes.

The radio-opacity for the calcium aluminate-based discs was considerably higher than for the calcium phosphate-based discs but lower than for the 2 mm thick Al discs.

### Conclusions

The calcium aluminate-based paste has a higher radio-opacity than the calcium phosphate-based paste, and considerably improved injectability.

1. A hydraulic ceramic precursor powder based on calcium aluminate and/or calcium silicate and zirconium and/or an inert filler material for orthopaedic and dental use, wherein the precursor powder has a water content below 0.08 weight-% measured as loss on ignition.

2. The hydraulic ceramic precursor according to claim 1, comprising:
55-65 wt-% of calcium aluminate,
35-45 wt-% of zirconium oxide and/or an inert filler material, and
0.5-5 wt-% of micro-silica,
wherein said components are based on the total amount of the precursor powder, and wherein the calcium aluminate is constituted by more than 70 atomic % of CaOAl₂O₃ and less than 30 atomic % of one or more of the phases (CaO)₁₂(Al₂O₃)₇, (CaO)₃Al₂O₃, CaO(Al₂O₃)₂, CaO(Al₂O₃)₆, and CaO—Al₂O₃ glass phase.

3. The precursor powder according to claim 1, wherein the powder comprises:
57-63 wt-% of calcium aluminate,
38-42 wt-% of zirconium oxide and/or an inert filler material, and
0.7-1.3 wt-% of micro-silica,
wherein said components are based on the total amount of the precursor powder, and wherein the calcium aluminate is constituted by more than 70 atomic % CaOAl₂O₃ and less than 30 atomic % of one or more of the phases (CaO)₁₂(Al₂O₃)₇, (CaO)₃Al₂O₃, CaO(Al₂O₃)₂, CaO(Al₂O₃)₆, and CaO—Al₂O₃ glass phase.

4. The precursor powder according to claim 1, wherein the calcium aluminate has a grain size of below 30 μm, the zirconium oxide a grain size of below 10 μm, and the micro-silica a grain size of below 30 nm.

5. The precursor powder according to claim 1, wherein the calcium aluminate has a grain size of below 15 μm, the zirconium oxide a grain size of below 5 μm, and the micro-silica a grain size of below 20 nm.

6. The precursor powder according to claim 1, wherein the calcium silicate, if present, comprises calcium silicate in the form of C₃S or C₂S, or combinations thereof, in an amount of less than 10 wt-% based on the total amount of the precursor powder.

7. The precursor powder according to claim 6, wherein the calcium silicate has a grain size of below 20 μm.

8. A hydration liquid for hydrating the precursor powder defined in claim 1 comprising:
90-95 wt-% of water,
3-5 wt-% of a compound based on polycarboxylic acid, and having a molecular weight of 10000-50000,
1-5 wt-% of methyl cellulose, and
less than 0.2 wt-% of LiCl,
wherein said amounts are based on the total weight of the hydration liquid.

9. The hydration liquid according to claim 8 wherein the hydration liquid comprises:
92-94 wt-% water,
3.7-4.3 wt-% of a compound based on polycarboxylic acid, and having a molecular weight of 10000-50000,
2.5-3.5 wt-% of methyl cellulose, and
0.05-0.2 wt-% of LiCl,
wherein said amounts are based on the total weight of the hydration liquid.

10. A ceramic paste comprising the precursor powder defined in claim 1 and a hydration liquid comprising
90-95 wt-% of water,
3-5 wt-% of a compound based on polycarboxylic acid, and having a molecular weight of 10000-50000,
1-5 wt-% of methyl cellulose, and less than 0.2 wt-% of LiCl,
wherein said amounts are based on the total weight of the hydration liquid, in a powder-to-liquid ratio of 3.75-5.

11. The ceramic paste according to claim 10, wherein the powder-to-liquid ratio is 4-4.5.

12. The ceramic paste according to claim 10, wherein the paste is injectable through gauge 13 needles or larger.

13. A method of manufacturing a chemically bonded ceramic material, comprising the step of mixing the precursor powder defined in claim 1 with a hydration liquid comprising
90-95 wt-% of water,
3-5 wt-% of a compound based on polycarboxylic acid, and having a molecular weight of 10000-50000,
1-5 wt-% of methyl cellulose, and
less than 0.2 wt-% of LiCl,
wherein said amounts are based on the total weight of the hydration liquid, in a liquid-to-powder ratio of 3.75-5.

14. The method according to claim 13, wherein the liquid-to-powder ratio is 4-4.5.

15. A chemically bonded ceramic material orthopaedic and dental applications, wherein said material is based on the precursor powder defined in claim 1, in hydrated form.

16. The material according to claim 15, wherein said material exhibits a total dimensional change of the material during setting and curing below +0.5 linear percent, an expansion of 0-0.5 linear percent, and/or exerts a total expansion pressure below 4 MPa on the environment.

17. A bioelement or implant for orthopaedic and dental applications, wherein said element is based on the precursor powder defined in claim 1.

18. A drug delivery carrier material, wherein said carrier material is based on the precursor powder defined claim 1.

19. A kit for manufacturing a chemically bonded ceramic material, comprising a container wherein the precursor powder defined in claim 1 and a hydration liquid comprising
90-95 wt-% of water,
3-5 wt-% of a compound based on polycarboxylic acid, and having a molecular weight of 10000-50000,
1-5 wt-% of methyl cellulose, and less than 0.2 wt-% of LiCl,
wherein said amounts are based on the total weight of the hydration liquid, are stored separately.

20. A kit according to claim 19, wherein the part of the container that holds the precursor powder exhibits a relative humidity (RH) of below 60%.

21. A kit according to claim 19, wherein the container that holds the precursor powder comprises vacuum and/or inert gas.
